(19) United States
(12) Patent Application Publication
(10) Pub. No.: US 2009/0112202 A1
(43) Pub. Date: Apr. 30, 2009

(54) DISPERSIVE ELECTRODE WITH THERMOCHROMATIC PROPERTIES

(75) Inventor: Kimbolt Young, Newtonville, MA (US)

Correspondence Address: Vista IP Law Group LLP, 2040 MAIN STREET, 9TH FLOOR, IRVINE, CA, 92614, US

(73) Assignee: BOSTON SCIENTIFIC SCIMED, INC., Maple Grove, MN (US)

(21) Appl. No.: 12/260,018

(22) Filed: Oct. 28, 2008

Related U.S. Application Data
(60) Provisional application No. 60/984,351, filed on Oct. 31, 2007.

(51) Int. Cl. A61B 18/18; A61B 18/14

(52) U.S. Cl. 606/33; 606/41

(57) ABSTRACT

A tissue treatment system includes an ablation energy generator, a treatment probe, and a dispersive electrode having a thermochromatic material that changes appearance upon reaching a predetermined temperature.

### RELATED APPLICATION

This Application claims priority to U.S. Provisional Patent Application No. 60/984,351 filed on Oct. 31, 2007. The above-noted Patent Application is incorporated by reference as if set forth fully herein.

### FIELD OF THE INVENTION

The field of the invention relates generally to the structure and use of tissue treatment systems, and in particular systems employing dispersive electrodes attached to body tissue for the treatment of tissue using electrical energy.

### BACKGROUND

The delivery of ablation energy, such as RF energy, to target regions within solid tissue is known for a variety of purposes of particular interest to the present invention. In one particular application, RF energy may be delivered to diseased regions (e.g., tumors) for the purpose of ablating predictable volumes of tissue with minimal patient trauma.

RF ablation of tumors is currently performed using one of two core technologies. The first technology uses a single needle electrode, which when attached to a RF generator, emits RF energy from an exposed, un-insulated portion of the electrode. The second technology utilizes multiple needle electrodes, which have been designed for the treatment and necrosis of tumors in the liver and other solid tissues. U.S. Pat. No. 6,379,353 discloses such a probe, referred to as a LeVeen Needle Electrode™, which comprises a cannula and an electrode deployment member reciprocally mounted within the delivery cannula to alternately deploy an electrode array from the cannula and retract the electrode array within the cannula. Using either of the two technologies, the energy that is conveyed from the electrode(s) translates into ion agitation, which is converted into heat and induces cellular death via coagulation necrosis. The ablation probes of both technologies are typically designed to be percutaneously introduced into a patient in order to ablate the target tissue.

In one design of such ablation probes, RF current is delivered from an RF generator to the ablation probe in a monopolar fashion, which may be applicable to either of the two technologies. In such embodiments, the ablation probe includes an ablation electrode located on a distal tip of the ablation probe configured to deliver the RF energy to tissue targeted for ablation, and a dispersive electrode located remotely from the ablation electrode. The dispersive electrode has a sufficiently large area, so that the RF current density is low and non-injurious to surrounding tissue, and may be attached to the patient, preferably externally to the patient. The dispersive electrode receives the monopolar RF current that is delivered to the target tissue site by the ablation electrode, so that the RF current is safely removed from the patient and returned to the RF generator.

As the dispersive electrode continues to receive the RF current, its temperature increases. The dispersive electrode should be removed from contact with the patient's body before reaching a temperature which may harm the patient, i.e., burning the patient. As a guideline example, according to the National Burn Victim Foundation, an adult may acquire a third-degree burn in thirty-five seconds from contacting a substrate or material at 130° F., or in two minutes from contacting a substrate or material at 125° F. Children may acquire third degree burns at these temperatures levels in a shorter time period. Second-degree burns may also be experienced by adult or child patients at lower temperatures, or at the same temperatures in a lesser period of time.

To monitor the temperature of the dispersive electrode, typically the operating room nurse or other medical personnel touches the dispersive electrode at chosen intervals. When the nurse considers the dispersive electrode to be too hot to contact the patient, based on how the dispersive electrode feels to the nurse, the nurse may decide to remove the dispersive electrode from the patient. However, this determination is arbitrary based on how often the nurse touches the dispersive electrode and how the particular nurse reacts to various temperature levels.

Whether the dispersive electrode is removed also may depend on the stage of ablation at the target tissue site. For example, if the nurse touches the dispersive electrode and determines that it may be too hot to continue contacting the patient, the other medical personnel performing the ablation procedure may have to decide whether to continue with the ablation procedure if the target tissue has not yet been fully ablated, while risking harm to the patient due to the dispersive electrode temperature, or to cease the ablation procedure when the target tissue may not be fully ablated. In this situation, if the medical personnel performing the ablation procedure had advance notice that the dispersive electrode was reaching a temperature that could harm the patient, steps could have been taken to expedite the ablation procedure, to add cooling pads, or to temporarily cease the ablation procedure until the dispersive electrode temperature returned to a safer temperature level.

Therefore, there is a need in the art for an ablation system that allows a user to more accurately determine the temperature of a dispersive electrode during an ablation procedure. There is also a need in the art for an ablation system that provides a user with timely notice that a dispersive electrode is reaching a temperature at which the dispersive electrode should be removed from a patient to avoid harming the patient.

### SUMMARY

In accordance with a first aspect of the present inventions, a tissue treatment system is provided. The system comprises a tissue treatment energy generator, a tissue treatment probe with an electrode, and a dispersive electrode. In particular, the system is a tissue ablation system with a tissue ablation energy generator, a tissue ablation probe with an ablation electrode, and a dispersive electrode. The tissue ablation energy is delivered from the generator to the ablation probe in a monopolar fashion to ablate target tissue in a patient. The dispersive electrode is placed in contact with the patient and receives the ablation energy as it passes from the ablation probe through the target tissue. The dispersive electrode returns the ablation energy to the generator, which causes the temperature of the dispersive electrode to increase.

The dispersive electrode has at least one thermochromatic material carried thereon or therein that changes appearance upon reaching a predetermined temperature. The thermochromatic material is preferably carried on the dispersive electrode to be visible during a tissue treatment procedure, so that a change in appearance of the thermochromatic material may be readily observed.

In one embodiment, the predetermined temperature may correspond to a temperature at which the patient may be burned from continued contact with the dispersive electrode. In this manner, the change in appearance of the thermochromatic material indicates that patient contact with the dispersive electrode should cease immediately or within a short period of time. In another embodiment, the predetermined temperature may correspond to a temperature lower than that at which the patient may be burned from continued contact with the dispersive electrode. In this manner, the change in appearance of the thermochromatic material indicates that patient contact with the dispersive electrode should cease before an extended period of time.

The thermochromatic material may be liquid crystals, a leucodye, or any material known in the art to change appearance at a predetermined temperature. In the liquid crystal form, the thermochromatic material may be carried on the dispersive electrode on an intermediary disposed over the electrode, such as a strip, or the thermochromatic material may be directly applied over a surface of the electrode, as examples. In the leucodye form, the thermochromatic material may be carried on the dispersive electrode in a compartment on the dispersive electrode or may be embedded in a surface of the dispersive electrode.

In one embodiment, the thermochromatic material may change appearance at the predetermined temperature by changing from a first color to a second color, or alternatively by changing from a first color to a transparent state. In another embodiment, the thermochromatic material may change appearance by changing from a first color to a second color in the form of a symbol or image. The symbol or image may be a visual graphic, symbol, or even text (e.g., words such as “HOT”).

The thermochromatic material may include first and second thermochromatic materials, wherein the first thermochromatic material changes appearance at a first predetermined temperature, and the second thermochromatic material changes appearance at a second, higher predetermined temperature. In this manner, the change in appearance of the first and second thermochromatic materials indicates different temperature levels of the dispersive electrode. In addition, the change in appearance of the first thermochromatic material may indicate that patient contact with the dispersive electrode should cease within an extended period of time, and the change in appearance of the second thermochromatic material may indicate that patient contact with the dispersive electrode should cease immediately or within a short period of time.

Methods of using the tissue treatment system are also provided. The methods comprise introducing the tissue treatment probe into the patient and delivering tissue treatment energy from the generator to the probe to treat the target tissue. As the dispersive electrode receives the tissue treatment energy, the dispersive electrode is observed for a change in appearance of the thermochromatic material, and in particular to determine if the temperature of the dispersive electrode is at or approaching a level at which the patient may be burned from continued contact with the dispersive electrode. After a change in appearance in the thermochromatic material is observed, delivery of the tissue treatment energy ceases and the dispersive electrode is removed from contacting the patient.

Other and further aspects and features of the invention will be evident from reading the following detailed description of the preferred embodiments, which are intended to illustrate, not limit, the present inventions.

### BRIEF DESCRIPTION OF THE DRAWINGS

Embodiments of the invention are illustrated byway of example and not limitation in the figures of the accompanying drawings, in which like references indicate similar elements, and in which:

FIG. 1 is a plan view of a tissue treatment system arranged in accordance with one embodiment of the present inventions.

FIGS. 2A and 2B are perspective views of alternative embodiments of a dispersive electrode that can be used in the tissue treatment system of FIG. 1.

FIG. 3 is a perspective view of another embodiment of a dispersive electrode that can be used in the tissue treatment system of FIG. 1.

FIG. 4 is a perspective view of another embodiment of a dispersive electrode that can be used in the tissue treatment system of FIG. 1.

FIGS. 5A and 5B are perspective views of another embodiment of a dispersive electrode that can be used in the tissue treatment system of FIG. 1, featuring a thermochromatic material changing appearance.

FIGS. 6A and 6B are perspective views of another embodiment of a dispersive electrode that can be used in the tissue treatment system of FIG. 1, featuring a thermochromatic material changing appearance.

FIGS. 7A-7C are perspective views of another embodiment of a dispersive electrode that can be used in the tissue treatment system of FIG. 1, featuring multiple thermochromatic materials changing appearance.

FIGS. 8A and 8B are perspective views of another embodiment of a dispersive electrode that can be used in the tissue treatment system of FIG. 1, featuring multiple thermochromatic materials changing appearance.

FIGS. 9A and 9B illustrate combined side and cross-sectional views of one method of using the tissue ablation system of FIG. 1 to treat tissue.

FIGS. 10A-10C illustrate combined side and cross-sectional views of another method of using the tissue ablation system of FIG. 1 to treat tissue.

### DETAILED DESCRIPTION OF THE ILLUSTRATED EMBODIMENTS

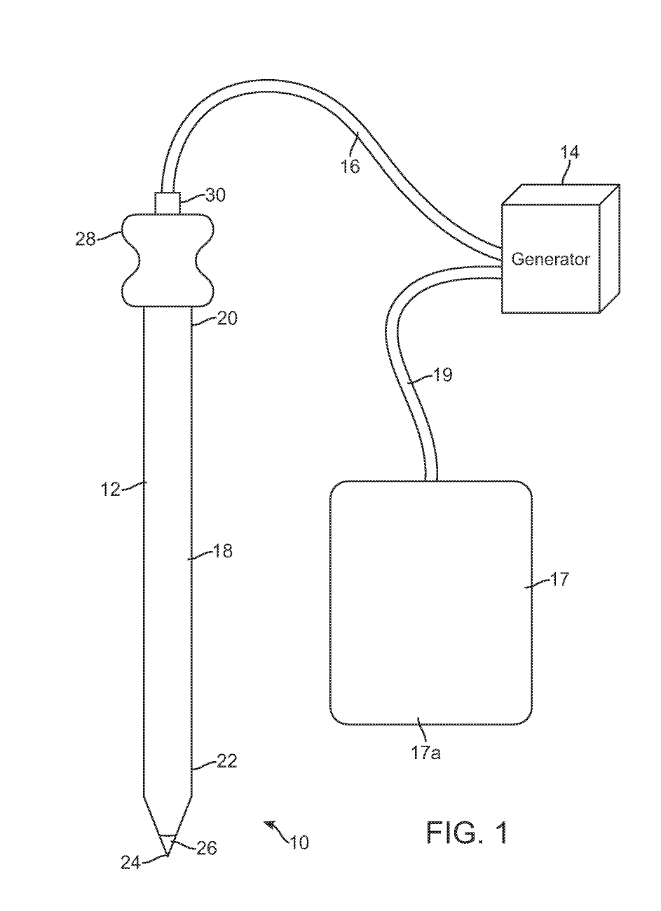

Referring to FIG. 1, a tissue treatment system 10 constructed in accordance with one embodiment of the present inventions, will now be described. The tissue treatment system 10 generally comprises: a tissue treatment probe 12, in particular an ablation probe 12, configured for introduction into the body of a patient for treatment of target tissue; a source or generator 14 of tissue treatment energy, in particular an ablation energy generator 14; a delivery cable 16 electrically connecting the probe 12 to the generator 14; a dispersive electrode 17; and a return cable 19 electrically connecting the dispersive electrode 17 to the generator 14.

In the illustrated embodiment, the generator 14 is an RF generator 14 for delivering RF ablation energy. The ablation system 10 may employ various embodiments of the ablation probe 12. In the illustrated embodiment, the ablation probe 12 comprises an elongated, rigid probe shaft 18 having a proximal end 20 and a distal end 22. In alternative embodiments, the probe shaft 18 may be flexible for conforming to vessels and/or other tissue surfaces. The probe shaft 18 has a suitable length, typically in the range from 5 cm to 30 cm, preferably from 10 cm to 25 cm, and an outer diameter consistent with its intended use, typically being from 0.7 mm to 5 mm, usually from 1 mm to 4 mm. In the illustrated embodiment, the probe shaft 18 is composed of an electrically conductive material, such as stainless steel. The distal end 22 of the probe shaft 18 includes a tissue-penetrating distal tip 24 that allows the ablation probe 12 to be more easily introduced through tissue while minimizing tissue trauma.

The tissue ablation probe further comprises an electrode 26 carried on the distal end 22 of the probe 12 for application in a tissue treatment procedure, and in particular an ablation procedure. In the illustrated embodiment, the electrode 26 is an RF ablation electrode 26 formed by the distal tip 24. In alternative embodiments, the electrode 26 may be a discrete element that is mounted to the distal tip 24 via suitable means, such as bonding or welding.

The ablation probe 12 further comprises a handle 28 mounted to the proximal end 20 of the probe shaft 18. The handle 28 is preferably composed of a durable and rigid material, such as medical grade plastic, and is ergonomically molded to allow a physician to more easily manipulate the ablation probe 12. The handle 28 comprises an electrical connector 30 with which the delivery cable 16 mates. Alternatively, the delivery cable 16 may be hardwired within the handle 28. The electrical connector 30 is electrically coupled to the ablation electrode 26 via the probe shaft 18. Further details regarding electrode array-type and other probe arrangements are disclosed in U.S. Pat. No. 6,379,353 and U.S. application Ser. No. 11/456,034, which are incorporated herein by reference.

In the illustrated embodiment, the RF current is delivered to the ablation electrode 26 in a monopolar fashion, wherein the ablation electrode 26 in turn delivers the RF current to target tissue. The ablation electrode 26 is configured to concentrate the RF energy flux in order to have an injurious effect on the surrounding target tissue.

The dispersive electrode 17 receives the RF energy that passes from the target tissue site through the patient's body and returns the RF current to the generator 14 via the return cable 19. The passage of RF energy from the ablation electrode 26 to the dispersive electrode 17 minimizes or prevents RF energy build-up that may harm the patient. However, this passage of RF energy also causes the temperature of the dispersive electrode 17 to increase, such that continued contact between the patient and the dispersive electrode 17 could possibly burn the patient, when the dispersive electrode 17 is at a sufficiently high temperature.

The dispersive electrode 17 is located remotely from the ablation electrode 26 and has a sufficiently large patient contact surface 17a (typically 130 cm² for an adult) to be placed in contact with the patient. The large patient contact surface 17a lowers the RF current density and reduces potential harm to bodily tissue. To position the dispersive electrode 17 in contact with the patient, the dispersive electrode 17 may include an adhesive material. Alternatively, a strap or other tying device may be used.

The dispersive electrode 17 may further embody any of the various structures known in the art and is not limited to a particular structure. As a general example, the dispersive electrode 17 includes a conductive element 5 configured to be electrically connected to the return cable 19. The dispersive electrode 17 may also include a conductive intermediary 6, for example a conductive gel or cream, as an interface between the patient and the conductive element 5. To provide examples, in one embodiment, the conductive element 5 includes a metal electrode plate and the conductive intermediary 6 includes a conductive gel for contacting the patient and facilitating RF current delivery from the patient to the dispersive electrode 17. In another embodiment, a flexible sheet of paperboard or other sufficiently flexible material is coated with a conductive foil for direct placement on the patient. In yet another embodiment, the dispersive electrode 17 includes a metal plate as a top layer, an insulative material as a middle layer, and a conductive adhesive for contacting the patient as a lower layer. In another embodiment, the dispersive electrode 17 includes a layer of conductive fibers in a mesh arrangement. This embodiment may also include an adhesive layer that is configured to adhere the conductive mesh to the patient. In yet another embodiment, the dispersive electrode 17 includes a flexible metalized plastic pad for direct placement on the patient.

The dispersive electrode 17 may also embody any of the electrical- and heat-transfer characteristics known in the art and is not limited to any particular electrical- and heat-transfer characteristics. For example, the dispersive electrode 17 may be a resistive-contact electrode, a capacitive-contact electrode, or a hybrid of the two.

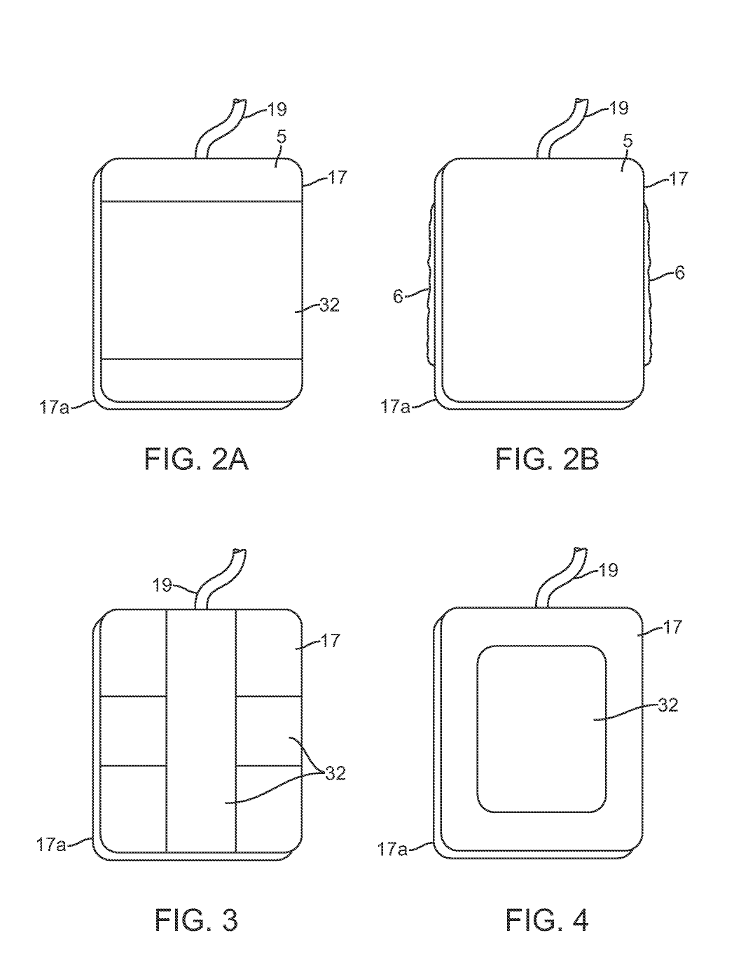

The dispersive electrode 17 includes a thermochromatic material 32 carried thereon that is calibrated to change appearance upon reaching a predetermined temperature. FIG. 2A illustrates the thermochromatic material 32 carried by a surface of the conductive element 5 of the dispersive electrode 17, and FIG. 2B illustrates the thermochromatic material 32 carried by the conductive intermediary 6 of the dispersive electrode, e.g. a conductive gel. As the temperature of the dispersive electrode 17 increases by receiving the RF energy, the temperature of the thermochromatic material 32 likewise increases and may reach or surpass the predetermined temperature, upon which the thermochromatic material 32 changes appearance.

The predetermined temperature at which the thermochromatic material 32 changes appearance may vary by calibrating the thermochromatic material 32 as desired. In one embodiment, the thermochromatic material 32 is calibrated to change appearance at a predetermined temperature approximately corresponding to a temperature at which the dispersive electrode 17 may burn the patient upon continued contact between the patient and the dispersive electrode 17. Thus, the change in appearance of the thermochromatic material 32 indicates that the patient may be subject to burns from the dispersive electrode 17, if the dispersive electrode 17 is not removed from the patient either immediately or within a short period of time, or alternatively if some type of cooling agent such as ice packs or wet gauze is applied. In this manner, the change in appearance of the thermochromatic material 32 serves as a visual indicator of temperature of the dispersive electrode 17. The manner in which the thermochromatic material 32 changes appearance may include a change in color or other features, which will be described later in more detail.

The predetermined temperature at which the thermochromatic material 32 is calibrated to change appearance may be based on the type of signal or warning desired. For example, when the dispersive electrode 17 temperature is in the range of approximately 115° F. to 120° F., a patient may experience second degree burns from continued contact with the dispersive electrode 17, possibly in two minutes or less. As another example, when the dispersive electrode 17 temperature is in the range of approximately 120° F. to 130° F., a patient may experience third degree burns from continued contact with the dispersive electrode 17, possibly in two minutes or less.

Thus, in one embodiment, the thermochromatic material 32 may be calibrated to change appearance at a predetermined temperature in the range of approximately 115° F. to 135° F. to indicate that the patient may be burned from continued contact with the dispersive electrode 17. In another embodiment, the predetermined temperature is in the range of approximately 120° F. to 130° F. In another embodiment, the predetermined temperature is in the range of approximately 123° F. to 127° F.

It may also be desirable for the predetermined temperature to be below the approximate temperature at which the patient may be burned by the dispersive electrode 17. In this manner, the change in appearance of the thermochromatic material 32 serves as an advance signal or warning that the patient could be burned if contact with the dispersive electrode 17 continues for an extended period, for example, two or more minutes.

Thus, in one embodiment, the predetermined temperature may be in the range of approximately 90° F. to 130° F. In another embodiment, the predetermined temperature is in the range of approximately 100° F. to 120° F. In another embodiment, the predetermined temperature is in the range of approximately 105° F. to 115° F. In another embodiment, the predetermined temperature is in the range of approximately 108° F. to 112° F. The predetermined temperature may be within an even lower range, for example upper and lower range limits of 5° F. lower or more, if the system 10 is to be used for child patients, who have more sensitive skin.

Preferably, the thermochromatic material 32 is carried on a portion of the dispersive electrode 17 that is visible during an ablation procedure, particularly when the dispersive electrode 17 is placed in contact with the patient. For example, FIG. 2 illustrates the thermochromatic material 32 carried on a surface of the dispersive electrode 17 opposite the patient contact surface 17a. As another example, if the dispersive electrode 17 has one or more side surfaces visible during an ablation procedure, such as when the dispersive electrode 17 is positioned beneath the patient, then the thermochromatic material 32 may be carried on one or more side surfaces of the dispersive electrode 17 that is at least substantially perpendicular to the patient contact surface 17a.

While it is desirable that the thermochromatic material 32 is carried on the dispersive electrode 17 to be visible during an ablation procedure, it is also desirable for the thermochromatic material 32 to be carried on the dispersive electrode 17 where a significant portion of heat from the RF current will be present. This may depend on the electrical- and heat-transfer characteristics of the dispersive electrode 17. For example, it is known in the art that capacitive-contact dispersive electrodes 17 distribute the RF current more uniformly over the surface of the dispersive electrode 17. Thus, in an embodiment having a capacitive-contact dispersive electrode 17, it may be preferable for the thermochromatic material 32 to be carried over a central portion of the dispersive electrode 17. As another example, it is known in the art that resistive-contact dispersive electrodes 17 distribute the RF current so that current density is higher at the edges of the electrode 17 surface. Thus, in an embodiment having a resistive-contact dispersive electrode 17, it may be preferable for the thermochromatic material 32 to be carried over one or more edges of the dispersive electrode 17. If possible, the thermochromatic material 32 may be positioned over at least a substantial portion or an entire surface of the dispersive electrode 17 that is visible during an ablation procedure, to ensure that all “hot spots” will be indicated by the thermochromatic material 32.

The thermochromatic material 32 may consist of one or more of a variety of different materials that are known in the art to be capable of changing appearance at a predetermined temperature. In one embodiment, the thermochromatic material 32 includes liquid crystals 32. Liquid crystals 32 twist in response to changes in temperature, such that the colors reflected or absorbed by the liquid crystals 32 also change, as is known in the art. As a result, the liquid crystals 32 appear to change color, or more specifically, the liquid crystals 32 appear to change from a first color to a second color.

Different forms of liquid crystals 32 are also known in the art, and the liquid crystals 32 may be carried on the dispersive electrode 17 in any form suitable for the purpose of the invention. In one embodiment, the liquid crystals 32 are carried on an intermediary that is in turn carried on the dispersive electrode 17. For example, the liquid crystals 32 may be sprayed on one or more flat strips and covered with a protective coating, wherein the one or more strips are carried on the dispersive electrode 17 so as to be visible during an ablation procedure, as shown in FIG. 3. In another embodiment, the liquid crystals 32 are applied directly to a surface of the dispersive electrode, for example by spraying or painting the liquid crystals 32 onto the dispersive electrode 17, as shown in FIG. 2.

As an alternative to the liquid crystals 32, the thermochromatic material 32 may also be a leucodye 32. Leucodyes 32 typically transition from having a first color to becoming transparent upon reaching a predetermined temperature, as is known in the art. The leucodye 32 may have a liquid or gel form, either of which may be contained in a compartment (not shown) carried by the dispersive electrode 17. In an alternative embodiment, the leucodye 32 may be combined with a substrate to create a solid leucodye form 32 that is carried by the dispersive electrode 17. The solid leucodye form 32 may be carried on a surface of the dispersive electrode 17. Alternatively, the solid leucodye form 32 may be embedded in a surface of the dispersive electrode 17, as shown in FIG. 4, preferably in a manner that will not impede conductivity of the RF energy through the dispersive electrode. This embodiment may be used when it is desired to avoid having a liquid or gel on the dispersive electrode 17 that could possibly run or leak and disrupt an ablation procedure. Substrates that may be incorporated in the solid leucodye form 32 include plastics, elastomers, or other suitable materials. Plastic and elastomeric forming processes that include the addition of a dye are well-known in the art, and any such process capable of producing the solid leucodye form 32 may be used.

In another embodiment, the leucodye 32 may be incorporated in the conductive intermediary 6 (see FIG. 2B) interfaced between the patient and the conductive element 5 of the dispersive electrode 17. In this embodiment, a portion or the entirety of the conductive element 5 may be substantially transparent, such that color changes of the leucodye 32 are readily viewable.

Because the leucodye 32 typically becomes transparent upon reaching a predetermined temperature, the leucodye 32 may be combined with a color-constant dye to more readily display the change in appearance of the leucodye 32. To illustrate, a leucodye 32 that is blue at room temperature combines with a yellow color-constant dye to create a combined dye appearing green at room temperature. Upon reaching the predetermined temperature, the leucodye 32 transitions from blue to transparent, while the color-constant dye remains yellow, so the combined dye appears to change appearance from green to yellow.

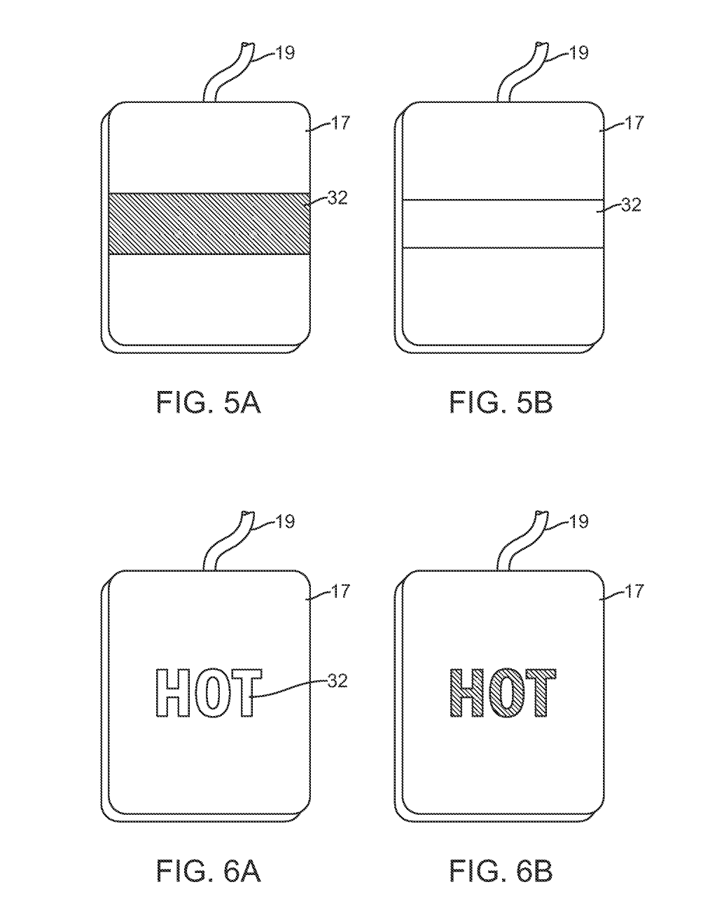

To describe how the thermochromatic material 32 may change appearance at the predetermined temperature, in one embodiment, the thermochromatic material 32 appears on the dispersive electrode 17 as having a first color at room temperature, as shown in FIG. 5A. As the temperature of the dispersive electrode 17 increases and the thermochromatic material 32 reaches the predetermined temperature, the appearance of the thermochromatic material 32 changes from the first color to a second color, as shown in FIG. 5B. The second color may appear as a new color, e.g., in the liquid crystal 32 embodiment of the thermochromatic material 32. As another example, the second color may appear as transparent or having no color, e.g., in the leucodye 32 embodiment of the thermochromatic material 32. Alternatively, if the leucodye 32 is combined with a color-constant dye, the leucodye 32 becomes transparent so that only the color of the color-constant dye remains visible, i.e. as the second color.

The change in appearance of the thermochromatic material 32 may also feature other visual indicators. For example, below the predetermined temperature, the thermochromatic material 32 may appear as having a first color and be applied or affixed to the dispersive electrode 17 in the form of an image or symbol. For example, as shown in FIG. 6A, the thermochromatic material 32 may be affixed to the dispersive electrode 17 in the form of the word “HOT,” and have a first color blue. When the thermochromatic material 32 reaches the predetermined temperature, as shown in FIG. 6B, the thermochromatic material 32 changes appearance by changing from the first color blue to a second color, such as red, wherein the word “HOT” appears red. In a similar example, the leucodye 32 may be affixed to the dispersive electrode 17 in the form of the word “HOT” and combined with a color-constant dye, such as a red dye. When the leucodye 32 reaches the predetermined temperature, the leucodye 32 turns transparent, while the color-constant dye remains red, so that “HOT” appears red. Other symbols, graphics, and images, as well as other first and second colors, may also be contemplated.

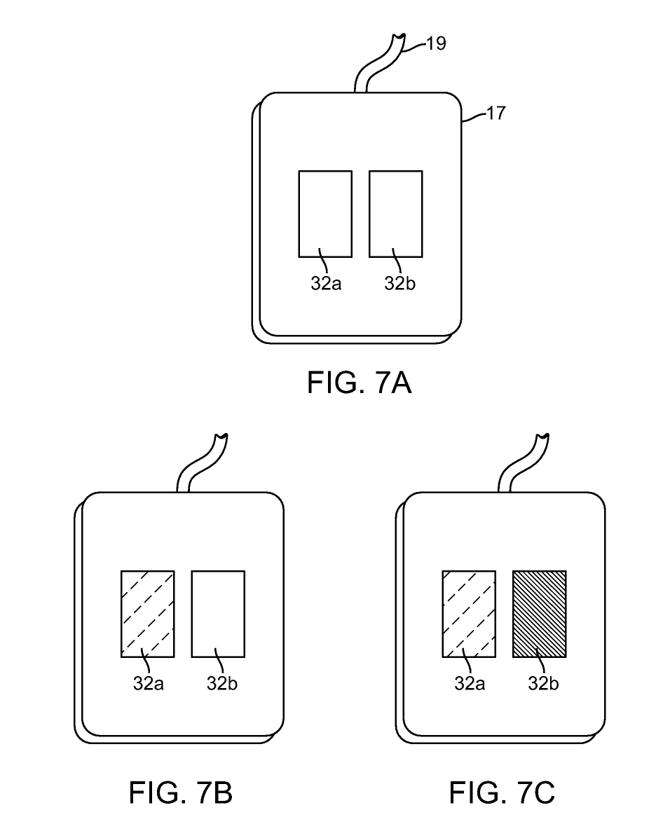

In another embodiment, the thermochromatic material 32 may consist of two or more thermochromatic materials 32a, 32b that change appearance at different predetermined temperatures. For example, a first thermochromatic material 32a that changes appearance at a first predetermined temperature may be combined with a second thermochromatic material 32b that changes appearance at a second predetermined temperature, wherein the second predetermined temperature is higher than the first predetermined temperature. As an alternative to combining the first and second thermochromatic materials 32a, 32b, the first and second thermochromatic materials 32a, 32b may be carried on the dispersive electrode 17 in separate locations. For example, the first and second thermochromatic materials 32a, 32b may be located adjacent or proximate to each other on the dispersive electrode 17, as shown in FIGS. 7A-7C.

To describe the change in appearance of the first and second thermochromatic materials 32a, 32b, the first thermochromatic material 32a may have a first color below the first predetermined temperature, and the second thermochromatic material 32b may also have a first color below the second predetermined temperature. In the embodiment in which the first thermochromatic material 32a and the second thermochromatic material 32b are combined, the first colors of both the first and second thermochromatic materials 32a, 32b are preferably the same. In the embodiment in which the first and second thermochromatic materials 32a, 32b are separate, the first colors of the first and second thermochromatic materials 32a, 32b may be the same, as shown in FIG. 7A, or different.

When the first thermochromatic material 32a reaches the first predetermined temperature, the first thermochromatic material 32a changes from its first color to a second color, while the second thermochromatic material 32b remains the same, as shown in FIG. 7B. As the temperature of the dispersive electrode 17 increases, the second thermochromatic material 32b reaches the higher second predetermined temperature and changes from its first color to a second color, as shown in FIG. 7C. Preferably, the second colors for each of the first and second thermochromatic materials 32a, 32b are different from each other, as shown in FIG. 7C, such that the respective second colors of each of the first and second thermochromatic materials 32a, 32b may be readily distinguishable.

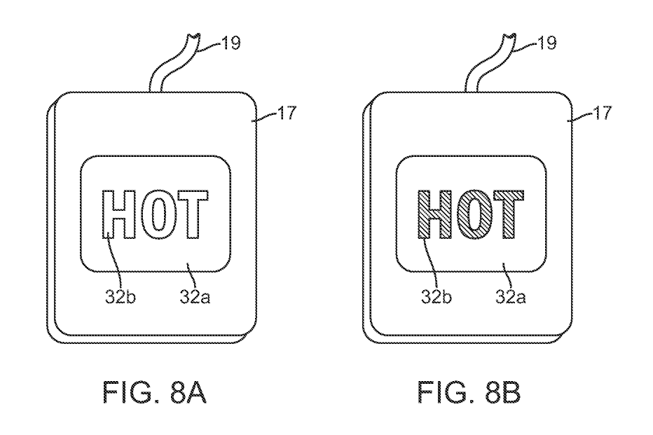

In an alternative embodiment, the first thermochromatic material 32a may change appearance at a first predetermined temperature by changing from a first color to a second color. The second thermochromatic material 32b may change appearance at a second predetermined temperature by changing from a first color to a second color shown in an image or symbol. Preferably, the second colors for each of the first and second thermochromatic materials 32a, 32b are different from each other, such that the respective second colors of each of the first and second thermochromatic materials 32a, 32b, and in particular the symbol or image, may be readily distinguishable. For example, referring to FIG. 8A, the first thermochromatic material 32a may have a first color yellow and change to a second color orange at the first predetermined temperature. The second thermochromatic material 32b may be in the form of the word “HOT” and have a first color blue. Referring to FIG. 8B, upon reaching the second predetermined temperature, which is higher than the first predetermined temperature, the second thermochromatic material changes from the first color blue to a second color red, so that “HOT” appears red.

The embodiment with the first and second thermochromatic materials 32a, 32b may further serve as a warning system. More specifically, the first predetermined temperature may be lower than the temperature at which the patient may be burned by continued contact with the dispersive electrode 17, and the second predetermined temperature may correspond approximately to a temperature at which the dispersive electrode 17 may burn the patient upon continued contact with the dispersive electrode 17. For example, the first predetermined temperature may be in the range of approximately 90° F. to 120° F., and the second predetermined temperature may be in the range of approximately 120° F. to 150° F. In another embodiment, the first predetermined temperature is in the range of 100° F. to 120° F., and the second predetermined temperature is in the range of 120° F. to 140° F. In another embodiment, the first predetermined temperature is in the range of 110° F. to 120° F., and the second predetermined temperature is in the range of 120° F. to 130° F. In this manner, the change in appearance of the first thermochromatic material 32a indicates that the dispersive electrode 17 is approaching a temperature at which the patient may be burned if contact with the dispersive electrode 17 is continued for an extended period of time. In addition, the change in appearance of the second thermochromatic material 32b serves as a more urgent alert that the patient may be burned if the dispersive electrode 17 is not removed from contacting the patient immediately or within a short period of time.

While the above-illustrated embodiments describe a tissue ablation system 10, other embodiments of the tissue treatment system 10 may be contemplated for other types of treatment. For example, the tissue treatment system 10 may comprise an electrosection system 10 for cutting tissue. For this and other embodiments of the system 10, the tissue treatment energy may be any energy that is suited to the type of treatment to be applied by the system 10.

In the embodiment for which the system 10 includes an RF generator 14, the RF generator 14 may be a conventional general purpose electrosurgical power supply operating at a frequency in the range from 300 kHz to 9.5 MHz, with a conventional sinusoidal or non-sinusoidal wave form. Such power supplies are available from many commercial suppliers, such as Valleylab, Aspen, Bovie, and Ellman. Most general purpose electrosurgical power supplies, however, are constant current, variable voltage devices and operate at higher voltages and powers than would normally be necessary or suitable. Thus, such power supplies will usually be operated initially at the lower ends of their voltage and power capabilities, with voltage then being increased as necessary to maintain current flow. More suitable power supplies will be capable of supplying an ablation current at a relatively low fixed voltage, typically below 200 V (peak-to-peak). Such low voltage operation permits use of a power supply that will significantly and passively reduce output in response to impedance changes in the target tissue. The output will usually be from 5 W to 300 W, usually having a sinusoidal wave form, but other wave forms would also be acceptable. Power supplies capable of operating within these ranges are available from commercial vendors, such as Boston Scientific Therapeutics Corporation. Preferred power supplies are models RF-2000 and RF-3000, available from Boston Scientific Corporation.

Having described the structure of the tissue treatment system 10, its operation in treating targeted tissue will now be described. The treatment region may be located anywhere in the body where hyperthermic exposure may be beneficial. Most commonly, the treatment region will comprise a solid tumor within an organ of the body, such as the liver, kidney, pancreas, breast, prostrate, and the like. The volume to be treated will depend on the size of the tumor or other lesion, typically having a total volume from 1 cm³ to 150 cm³, and often from 2 cm³ to 35 cm³ The peripheral dimensions of the treatment region may be regular, e.g., spherical or ellipsoidal, but will more usually be irregular. The treatment region may be identified using conventional imaging techniques capable of elucidating a target tissue, e.g., tumor tissue, such as ultrasonic scanning, magnetic resonance imaging (MRI), computer-assisted tomography (CAT), fluoroscopy, nuclear scanning (using radiolabeled tumor-specific probes), and the like.

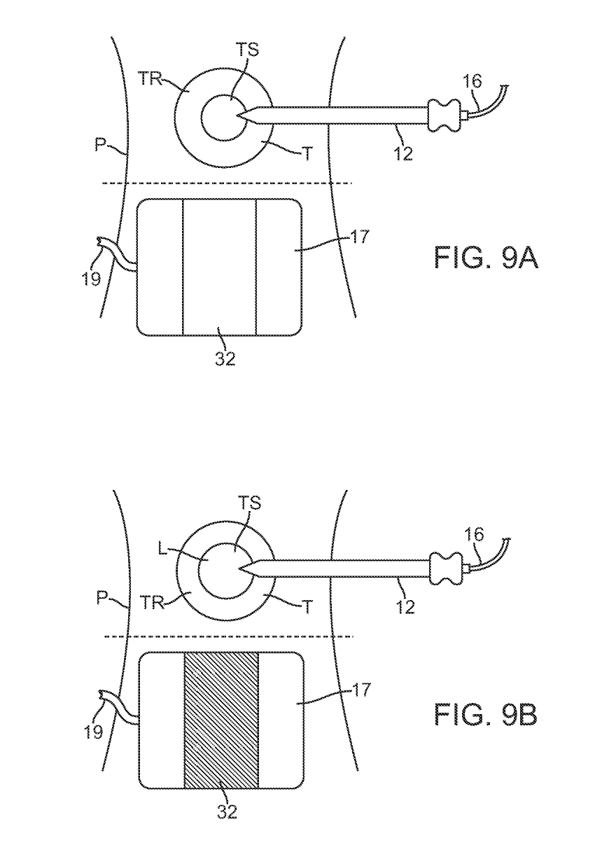

Referring now to FIGS. 9A and 9B, the operation of the tissue treatment system 10 is described in treating a treatment region TR with tissue T located beneath the skin of a patient P. For the illustrated embodiments, a method of using the tissue treatment system 10 for ablating tissue will be described. Facilitated by the sharpened distal tip 24, the ablation probe 12 is first introduced through the tissue T, so that the ablation electrode 26 is located at a target site TS within the treatment region TR, as shown in FIG. 9A. This can be accomplished using any one of a variety of techniques and devices that are known in the art, for example, using a conventional ultrasound imaging device. A probe guide (not shown) may also be used in cooperation with the ablation probe 12 to guide the probe 12 toward the target site TS.

In addition to positioning the ablation probe 12, the dispersive electrode 17 is placed on the patient such that at least a substantial portion of the patient contact surface 17a contacts the patient. The dispersive electrode 17 may be positioned in contact with the patient before, during, or after the ablation probe 26 is guided to the target site TS. However, as a safety measure it is desired that the dispersive electrode 17 is placed in contact with the patient before the ablation energy may be conducted to the patient. Preferably, any open gaps or spaces between the patient contact surface 17a and the patient are minimized or eliminated. Otherwise, the conductivity of the ablation energy to the dispersive electrode 17 may be inhibited, possibly harming the patient.

The dispersive electrode 17 may be positioned underneath the patient, for example, underneath the patient's thigh, such that the patient lies on top of the dispersive electrode 17. Alternatively, for the embodiment of the dispersive electrode 17 having an adhesive, the dispersive electrode 17 may be adhered to the patient, for example to the patient's thigh, hip, or buttocks. As another alternative, the dispersive electrode 17 may be tied with a strap or other device that substantially holds the dispersive electrode 17 in position. Preferably, the dispersive electrode 17 is positioned where any change in appearance of the thermochromatic material 32 may be readily observed.

Once the ablation probe 12 and the dispersive electrode 17 are properly positioned, the cable 16 of the RF generator 14 (shown in FIG. 1) is connected to the electrical connector 30 of the ablation probe 12. The RF generator 14 and probe 12 are then operated to deliver RF energy to the ablation electrode 26, thereby ablating the treatment region TR, as illustrated in FIG. 9B. As a result, a lesion L will be created, which will eventually expand to include the entire treatment region TR.

While the RF energy is delivered to the ablation electrode 26, the dispersive electrode 17 receives the RF energy as it passes from the target site TS through the patient. The dispersive electrode 17 then returns the RF energy to the generator 14 via the return cable 19 to reduce RF energy build-up in the patient. As the dispersive electrode 17 receives the RF energy, the dispersive electrode 17 temperature increases, as well as the thermochromatic material 32 temperature. To minimize or prevent burns to the patient resulting from contact with the dispersive electrode 17, the dispersive electrode 17 is observed for any change in appearance of the thermochromatic material 32. When the dispersive electrode 17 temperature increases such that the thermochromatic material 32 reaches the predetermined temperature, the thermochromatic material 32 changes appearance, as shown in FIG. 9B. Specifically, FIGS. 9A and 9B illustrate the embodiment in which the thermochromatic material 32 changes from a first color (FIG. 9A) to a second color (FIG. 9B).

For the embodiment in which the predetermined temperature approximately corresponds to a temperature at which the dispersive electrode 17 may burn the patient upon continued contact between the patient and the dispersive electrode 17, the change in appearance of the thermochromatic material 32 may signal the ablation system 10 users to discontinue delivery of the RF energy to the ablation electrode 26 immediately or within a short period of time, for example, within 35 seconds. The users may also apply a cooling agent to the region where the dispersive electrode 17 is located to prevent burning and possibly forestall ceasing delivery of the RF energy, if needed. Alternatively, for the embodiment in which the thermochromatic material 32 changes appearance at a predetermined temperature lower than that at which the patient may be burned by continued contact with the dispersive electrode 17, the change in appearance of the thermochromatic material 32 may signal the ablation system 10 users to discontinue delivery of the RF energy within a more extended period of time, for example, within two minutes. Also, the users may apply a cooling agent to the patient in the region where the dispersive electrode 17 is located.

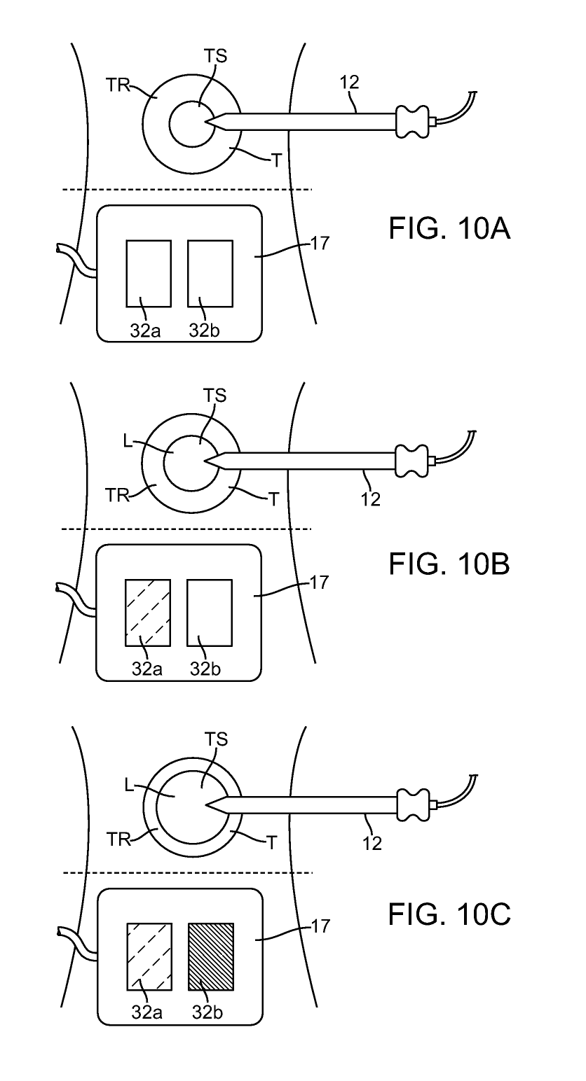

FIGS. 10A-10C illustrate an alternative method of using the system 10, in which the thermochromatic material 32 comprises a first thermochromatic material 32a and a second thermochromatic material 32b. FIGS. 10A and 10B illustrate the first thermochromatic material 32a changing from a first color (FIG. 10A) to a second color (FIG. 10B) upon reaching the first predetermined temperature. At the first predetermined temperature, the appearance of the second thermochromatic material 32b does not change. When the second thermochromatic material 32B reaches the second predetermined temperature, which is higher than the first predetermined temperature, the second thermochromatic material 32B changes from a first color (FIGS. 10A and 10B) to a second color (FIG. 10C). The change in appearance of the first thermochromatic material 32a may signal the ablation system 10 users to discontinue delivery of the RF energy to the ablation electrode 26 within a short period of time, for example, within two minutes. If the RF energy continues to be delivered to the ablation electrode 26, particularly to ensure ablation of the target tissue T, the change in appearance of the second thermochromatic material 32 may signal the ablation system 10 users to discontinue delivery of the RF energy immediately or within a short period of time, for example, within 35 seconds, or to apply a cooling agent.

After delivery of the RF energy to the ablation electrode 26 is discontinued, the dispersive electrode 17 is removed from contacting the patient. If the dispersive electrode 17 is removed before delivery of the RF energy is discontinued, RF energy may potentially build up in the patient and harm the patient. The ablation probe 12 is then removed from the target site TS.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A tissue ablation system, comprising:
a generator configured for delivering tissue ablation energy;
an ablation probe comprising an ablation energy electrode or antenna operatively coupled to the generator and configured for electrically coupling with body tissue to be ablated; and
a dispersive electrode operatively coupled to the generator and configured for electrically coupling with body tissue to complete an electrical circuit with the ablation energy electrode or antenna, the dispersive electrode comprising a thermochromatic material that changes appearance at a predetermined temperature.

2. The tissue ablation system of claim 1, wherein the dispersive electrode comprises a patient contact surface, and wherein the thermochromatic material is carried on a surface of the dispersive electrode opposite the patient contact surface.

3. The tissue ablation system of claim 1, wherein the dispersive electrode comprises a patient contact surface, and wherein the thermochromatic material is carried on a surface of the dispersive electrode substantially perpendicular to the patient contact surface.

4. The tissue ablation system of claim 1, wherein the thermochromatic material comprises liquid crystals.

5. The tissue ablation system of claim 4, wherein the liquid crystals are carried on an intermediary carried on the dispersive electrode.

6. The tissue ablation system of claim 1, wherein the thermochromatic material comprises a leucodye.

7. The tissue ablation system of claim 6, wherein the leucodye is embedded in a surface of the dispersive electrode.

8. The tissue treatment system of claim 1, wherein the thermochromatic material comprises liquid crystals.

9. The tissue ablation system of claim 8, wherein the liquid crystals are embedded in a surface of the dispersive electrode.

10. The tissue ablation system of claim 1, wherein the thermochromatic material changes appearance at the predetermined temperature by changing from a first color to a second color.

11. The tissue ablation system of claim 1, wherein the thermochromatic material changes appearance at the predetermined temperature by becoming substantially transparent.

12. The tissue ablation system of claim 1, wherein the thermochromatic material changes appearance at the predetermined temperature by showing an image.

13. The tissue ablation system of claim 1, wherein the predetermined temperature is in the range of approximately 90° F. to 130° F.

14. The tissue ablation system of claim 13, wherein the predetermined temperature is in the range of approximately 105° F. to 115° F.

15. The tissue ablation system of claim 14, wherein the predetermined temperature is in the range of approximately 108° F. to 112° F.

16. The tissue ablation system of claim 1, wherein the thermochromatic material comprises a first thermochromatic material and a second thermochromatic material, the first thermochromatic material calibrated to change appearance at a first predetermined temperature, and the second thermochromatic material calibrated to change appearance at a second predetermined temperature higher than the first predetermined temperature.

17. The tissue ablation system of claim 16, wherein the first predetermined temperature is in the range of approximately 90° F. to 120° F., and the second predetermined temperature is in the range of approximately 120° F. to 150° F.

18. The tissue ablation system of claim 17, wherein the first predetermined temperature is in the range of approximately 110° F. to 120° F., and the second predetermined temperature is in the range of approximately 120° F. to 130° F.

19. The tissue ablation system of claim 1, wherein the dispersive electrode comprises a metal plate.

20. The tissue ablation system of claim 1, wherein the dispersive electrode comprises conductive foil.

21. The tissue ablation system of claim 1, wherein the dispersive electrode comprises a conductive mesh.

22. The tissue ablation system of claim 1, wherein the dispersive electrode comprises a metalized plastic.

23. The tissue ablation system of claim 1, wherein the generator is a radiofrequency energy generator.

24. The tissue ablation system of claim 1, wherein the generator is a microwave energy generator.

25. A method of ablating body tissue, comprising:
locating an ablation element coupled to an active terminal of an energy generator proximate a region of internal body tissue to be ablated;
positioning a dispersive electrode on a surface of the body, the dispersive electrode coupled to a return terminal of the energy generator;
operating the generator to deliver ablation energy through the respective ablation element, body tissue, and dispersive electrode;
observing the dispersive electrode to detect a change in appearance of thermochromatic material carried on or in the dispersive electrode; and
discontinuing delivery of the ablation media to the ablation probe after detecting a change in appearance of the at least one thermochromatic material is observed.

26. The method of claim 25, wherein a change in appearance of the thermochromatic material comprises the thermochromatic material changing from a first color to a second color.

27. The method of claim 25, wherein a change in appearance of the thermochromatic material comprises the thermochromatic material becoming substantially transparent.

28. The method of claim 25, wherein a change in appearance of the thermochromatic material comprises the thermochromatic material showing a symbol.
